(19) United States
(12) Patent Application Publication
(10) Pub. No.: US 2020/0058400 A1
(43) Pub. Date: Feb. 20, 2020

(54) SYSTEM FOR UNDERSTANDING HEALTH-RELATED COMMUNICATIONS BETWEEN PATIENTS AND PROVIDERS

(71) Applicant: Patrick Leonard, Littleton, CO (US)

(72) Inventor: Patrick Leonard, Littleton, CO (US)

(21) Appl. No.: 16/554,404

(22) Filed: Aug. 28, 2019

Related U.S. Application Data
(63) Continuation of application No. 15/142,899, filed on Apr. 29, 2016, now abandoned.
(60) Provisional application No. 62/154,412, filed on Apr. 29, 2015.

(51) Int. Cl. G16H 50/20; G16H 10/60

(52) U.S. Cl. CPC G16H 50/20; H04L 67/22; G16H 10/60

(57) ABSTRACT

Systems, methods and apparatus are disclosed that provide an approach to understand, analyze and generate useful output of patient-provider interactions. Embodiments of the disclosure provide systems, methods and apparatus for creating understanding, and generating summaries and action item from an interaction between a patient, a provider and optionally a user.

### CROSS-REFERENCE TO RELATED APPLICATIONS

The present application is a continuation application of U.S. Ser. No. 15/142,899, filed Apr. 29, 2016, which claims benefit under 35 U.S.C. § 119(e) of U.S. Provisional Patent Application Ser. No. 62/154,412 entitled “SYSTEM FOR UNDERSTANDING HEALTH-RELATED COMMUNICATIONS BETWEEN PATIENTS AND PROVIDERS” filed on Apr. 29, 2015, the entire contents of each of the above-referenced applications are hereby expressly incorporated herein by reference.

### BACKGROUND

Studies indicate that patients have a very difficult time understanding and remembering what healthcare providers tell them during visits and other communications. One study from the National Institutes of Health (NIH) estimated that patients forget up to 80% of what was told to them in the doctor's office and misunderstand half of what they do remember. Understanding as little as 10-20% of what our healthcare providers tell us can have a serious negative impact on healthcare outcomes and costs.

The present disclosure is directed toward overcoming one or more of the problems discussed above.

### SUMMARY

Embodiments described in this disclosure provide systems, methods, and apparatus for listening and interpreting interactions, and generating useful medical information between at least one provider and at least one patient, and optionally a user.

Some embodiments provide methods, systems and apparatus of monitoring and understanding an interaction between at least one patient and at least one provider and optionally a user comprising: listening and/or observing the interaction; interpreting the interaction such as analyzing the interaction, wherein analyzing includes specific items from the interaction; and generating an output information that includes a summary of the interaction, and action to be taken by the patient and/or the provider in response to the specific item. These steps can be performed sequentially or in another order. In some embodiments, the interaction analyzed is between multiple parties such as a patient and more than one provider.

Some embodiments provide methods of monitoring and understanding an interaction between at least one patient and at least one provider and optionally a user comprising: (a) detecting the interaction between at least one patient and at least one provider and optionally at least one user; (b) receiving an input data stream from the interaction; (c) extracting the received input data stream to generate a raw information; (d) interpreting the raw information, wherein the interpretation comprises: converting the raw information using a conversion module to produce a processed information, and analyzing the processed information using an artificial intelligence module; (e) generating an output information for the interaction based upon the interpretation of the raw information comprising a summary of the interaction, and follow-up actions for the patient and/or provider; and (f) providing a computing device, the computing device performing steps “a” through “e”. In various embodiments of the methods disclosed herein, analyzing the processed information further comprises: understanding the content of the processed information; and optionally enriching the processed information with additional information from a database. Various embodiments of the methods disclosed herein further comprise the step of sharing the output information with at least the patient, the provider, and/or the user. Some embodiments of the methods disclosed herein further comprise the step of updating a patient record in an electronic health records system based upon the interpreted information or the output information. In some embodiments of the methods disclosed herein, the output information is further modified by the provider and/or optionally the user which can be shared with the patient, providers, and/or users. In some embodiments of the methods disclosed herein, the detection of the interaction is automatic or manually initiated by one of the provider, patient, or optionally user. The electronic health records system can be any system used in healthcare environment for maintaining all records related to the patient, provider, and/or optionally a user.

In some aspects, the interaction may be a conversation or one or more statements. In one embodiment, the conversion module comprises a speech recognition system. In some embodiments, the speech recognition system differentiates between the speakers, such as the patient and the provider.

In some embodiments, the output information is a summary of the interaction. In other embodiments, the output information is an action item for the patient and/or the provider to accomplish or perform. The action item includes, but is not limited to, a follow up appointment, a prescription for drugs or diagnostics, provider prescribed procedures for the patient without provider's supervision, provider prescribed another provider supervised medical procedures. In certain embodiments the output information comprises a summary of the interaction and action items for the patient and the provider.

The interaction between the patient and the provider may be in a healthcare environment. In the healthcare environment, the interaction may be a patient and/or provider conversation or statement. The healthcare environment can be physical location or a digital system. The digital system includes, but not limited to, teleconference, videoconference, or online chat.

Some embodiments disclosed herein provide a system comprising a computer memory storage module configured to store executable computer programming code; and a computer processor module operatively coupled to the computer memory storage module, wherein the computer processor module is configured to execute the computer programming code to perform the following operations: detecting an interaction between at least one patient and at least one provider and optionally at least one user; receiving an input data stream from the interaction; extracting the received input data stream to generate a raw information; interpreting the raw information, wherein the interpretation comprises: converting the raw information using a conversion module to produce a processed information, and analyzing the processed information using an artificial intelligence module; and generating an output information for the interaction based upon the interpretation of the raw information comprising a summary of the interaction, and follow-up actions for the patient and/or provider. In some embodiments of the disclosed system, analyzing the processed information further comprises: understanding the content of the processed information; and optionally enriching the processed information with additional information from a database. Some embodiments of the disclosed system, further comprises sharing the output information with at least one of the patient, the provider, and/or the user. Some embodiments of the system, further comprises updating a patient record in an electronic health records system based upon the interpreted information or the output information. In some embodiments of the disclosed system, the output information is modified by the provider and/or optionally the user. In some embodiments of the disclosed systems, the detection of the interaction is automatic or manually initiated by one of the provider, patient, or optionally user.

The input data stream can be in the form of input speech by the patient, the provider and/or the user. Yet another way the patient, the provider and/or the user generate input data stream is by inputting interaction such as via online chat or thoughts captured via brain-computer interface can be used in this step. These and other modes of conversation are simply a different input data stream, and the other embodiments of the system work the same. The input device used to generate the input data stream by the provider, the patient, and/or the user could be a microphone, keyboard, a touchscreen, a joystick, a mouse, a touchpad and/or a combination thereof.

Some embodiments provide an apparatus comprising a non-transitory, tangible machine-readable storage medium storing a computer program, wherein the computer program contains machine-readable instructions that when executed electronically by one or more computer processors, perform: detecting an interaction between at least one patient and at least one provider and optionally at least one user; receiving an input data stream from the interaction; extracting the received input data stream to generate a raw information; interpreting the raw information, wherein the interpretation comprises: converting the raw information using a conversion module to produce a processed information, and analyzing the processed information using an artificial intelligence module; and generating an output information for the interaction based upon the interpretation of the raw information comprising a summary of the interaction, and follow-up actions for the patient and/or provider. In some embodiments of the disclosed apparatus, analyzing the processed information further comprises: understanding the content of the processed information; and optionally enriching the processed information with additional information from a database. Some embodiments of the disclosed system further comprises sharing the output information with at least one of the patient, the provider, and/or the user. Some embodiments of the disclosed system further comprise updating a patient record in an electronic health records system based upon the interpreted information or the output information. In some embodiments of the disclosed apparatus, the output information is modified by the provider and/or optionally the user. In some embodiments of the disclosed system the detection of the interaction is automatic or manually initiated by one of the provider, patient, or optionally user.

### BRIEF DESCRIPTION OF THE DRAWINGS

FIG. 1 shows a pictorial view of the full system and major parts according to one embodiment of the present invention.

FIG. 2 shows a detail view of the Analyze & Extract step according to one embodiment of the present invention.

FIG. 3 shows a chronological flow diagram for the experience of people using embodiments of the disclosed system in one example of its operation.

FIG. 4 shows screen mockups of the user interface for several of the steps used in the operation of the system according to one embodiment of the present invention.

FIG. 5 shows a flow diagram for intents and entities according to one aspect of the disclosure.

### DESCRIPTION

Systems, methods, and apparatus are disclosed that comprise a combination of listening, and interpreting the information, generating summaries, and creating actions to facilitate understanding and actions from interactions between a patient and provider. The disclosed embodiments use various associated devices, running related applications and associated methodologies in implementing the system. The interaction herein can be conversational and/or include one or more statements.

As used herein, a “provider” is any person or a system providing health or wellness care to someone. This includes, but is not limited to, a doctor, nurse, physician's assistant, or a computer system that provides care. The provider in the “patient-provider” conversation does not have to be a human. The provider can also be an artificial intelligence systems, technology-enhanced humans, artificial life forms and genetically engineered life forms created to provide health and wellness services.

As used herein, a “patient” is a person receiving care from a provider, or a healthcare consumer, or other user of this system and owner of the data contained within. The patient in the “patient-provider” conversation also does not have to be a human. The patient can be animal, artificial intelligence systems, technology-enhanced humans, artificial life forms and genetically engineered life forms.

As used herein, a “user” is anyone interacting with any of the embodiments of the system. For example the user can be a caregiver, family member of the patient, friend of the patient, an advocate for the patient, an artificial intelligence system, technology-enhanced humans, artificial life forms and genetically engineered life forms or anyone or anything else capable of adding context to the interaction between a patient and a provider, or any person or system facilitating patient's communication with the provider. The advocate can be a traditional patient advocate but does not have to be a traditional patient advocate.

As used herein the “input data stream” is all forms of data generated from the interaction between patient and provider and/or user, including but not limited to, audio, video, or textual. The audio can be in any language.

The “raw information” as used herein refers to an exact replication of all input data stream from the patient, provider, and optionally a user interaction.

The conversion module comprises a speech recognition module capable of converting any language or a combination of languages in the raw information into a desired language. The conversion module is also configured to convert the raw information in the form of audio, video, textual or binary or a combination thereof into a processed information in a desired format that is useful for analysis by the artificial intelligence module. The artificial intelligence module can be configured to accept the processed information in any format such as audio, video, textual or binary or a combination thereof.

The term “sensing” herein refers to mechanisms configured to determine if a patient may be having or is about to have an interaction with their provider. Sensing when it is appropriate to listen can be done using techniques other than location and calendar. For example, beacons may be used to determine fine grained location. Or big data analytics can be used to mine data sets for patterns. Embodiments disclosed herein detect an interaction between at least one patient and at least one provider and optionally at least one user. The detection of the interaction can be automatic such as by sensing or it can be manually initiated by a provider, a patient or a user.

Some embodiments disclosed herein, and certain components thereof, listen to the interaction between a patient, a provider, and/or a user to generate raw information, and automatically interpret the raw information to generate an output information, that is useful and contextual. The output information may include a summary of the interaction, reminders, and other useful information and actions. The raw information from the interaction may be transferred in whole or in part. In addition to transmitting an entire raw information as a single unit, the raw information can be transferred in parts or in a continuous stream for interpretation.

Some embodiments disclosed herein, and certain components thereof may listen to interaction in which there are multiple parties and different streams of interaction.

In some embodiments, the raw information obtained from the interaction is further enriched with additional context, information and data from outside the interaction to make it more meaningful to generate an enriched raw information. Some embodiments use the enriched raw information for interpretation as disclosed herein to generate an output information from the enriched raw information.

In various embodiments, the output information can be viewed and/or modified (with permission) by the provider and/or the user to add or clarify output information so as to generate a modified output information.

In various embodiments, the raw information, the output information and the modified output information can be shared with other people, who may include family members, providers, other caregivers, and the like.

In various embodiments, the output information or the modified output information, is automatically generated after a patient's clinic visit and interaction with the provider.

In various embodiments, the output information or the modified output information, generates actions and/or reminders to improve the workflow of the provider's medical treatment operations. In an embodiment, the output information or the modified output information may initiate the patient's scheduling of a follow up appointment, diagnostic test or treatment.

In an embodiment, elements of the interaction are used to pre-populate medical coding applications to save time and to increase the accuracy of medical procedures and tests.

One advantage offered by embodiments herein is to provide patients with a deeper and/or greater understanding of what a provider was advising and/or informing a patient during their interaction.

Other advantages offered by embodiments herein allow for no note taking by patients and/or provider during the patient-provider interaction. Particularly, most patients do not take notes of their interactions with their providers, and those who do generally find it to be difficult, distracting and incomplete. The various embodiments disclosed herein will record the interaction between the patient and the provider, where notes of the interaction need not be maintained by the patient and/or the provider, and then the embodiments herein will generate an output information that comprises summary of the interaction in a format that is much more useful for later reference than having to replay an exact record of the whole interaction. The various embodiments disclosed herein can generate an output information from the interaction in various ways depending upon the desirability of the type of processing of the interaction. For example, either the patient or the provider can request the raw information, enriched raw information, output information, and/or modified output information.

Another advantage offered by one or more embodiments of the disclosed system is that the patients will have follow up reminders or “to-dos” created for them and made available on a mobile device such as a smart mobile device or a handheld computing device. These may include, but are not limited to, to-dos in a reminders list application or appointment entries in a calendar application. Most providers do not provide explicit instructions for patients and those who do generally put it on a piece of paper which may be lost or ignored. Automatically generating reminders and transmitting them to the patient's mobile device makes it easier and more likely that patients will do the things that they need to do as directed by their provider. This can have a significant positive impact on “adherence” or “patient compliance” by the patient, a major healthcare issue responsible for a massive amount of cost and poor health outcomes.

Another advantage offered by one or more embodiments of the disclosed system is the engagement of patient advocates (a third party who acts on behalf of the patient). Patient advocates can provide significant value to the health of a patient or healthcare consumer, but their services are currently available to only a small fraction of the population. Various embodiments of the disclosed system may remotely and automatically share the various system generated output information of the patient-provider engagement with patient advocates. The combination of remote access and automation provides a way for patient advocacy to be made available to a mass market with much lower cost and less logistical difficulty. For example, a patient diagnosed with diabetes would have the system generated output information that comprises appropriate information from the American Diabetes Association®.

Another advantage offered by one or more embodiments of the disclosed system is the ability to easily share information with family and other caregivers. The output information such as summaries, reminders and other generated information can be shared (with appropriate security and privacy controls) with other caregivers such as family, patient advocates or others as the patient desires. Very few people today have a good way to share this type of health information easily and securely.

Another advantage offered by one or more embodiments of the disclosed system is the detection (e.g. sensing) that a patient is likely in a situation where it makes sense to listen to the interaction between the patient and another party such as a provider. The detection reduces the need for the patient to remember to engage components of the system to start the listening process to capture their interaction. The less people have to think about using this type of system and its components the more likely they are to experience its benefits.

Another advantage offered by one or more embodiments of the disclosed system is the ability to capture interactions in which there are multiple parties and different streams of interactions. This enables the parties to have a regular interaction in addition to, or instead of the traditional provider dictation such as physician dictation of their notes. This multi-party interaction has information that the physician notes lack, including, but not limited to, information that the patient and/or their family possesses, questions asked by the patient and/or their family, responses from the physician and/or staff, information from specialist in consultation with the physician and/or staff, sentiments and/or emotions conveyed by the patient and/or their family.

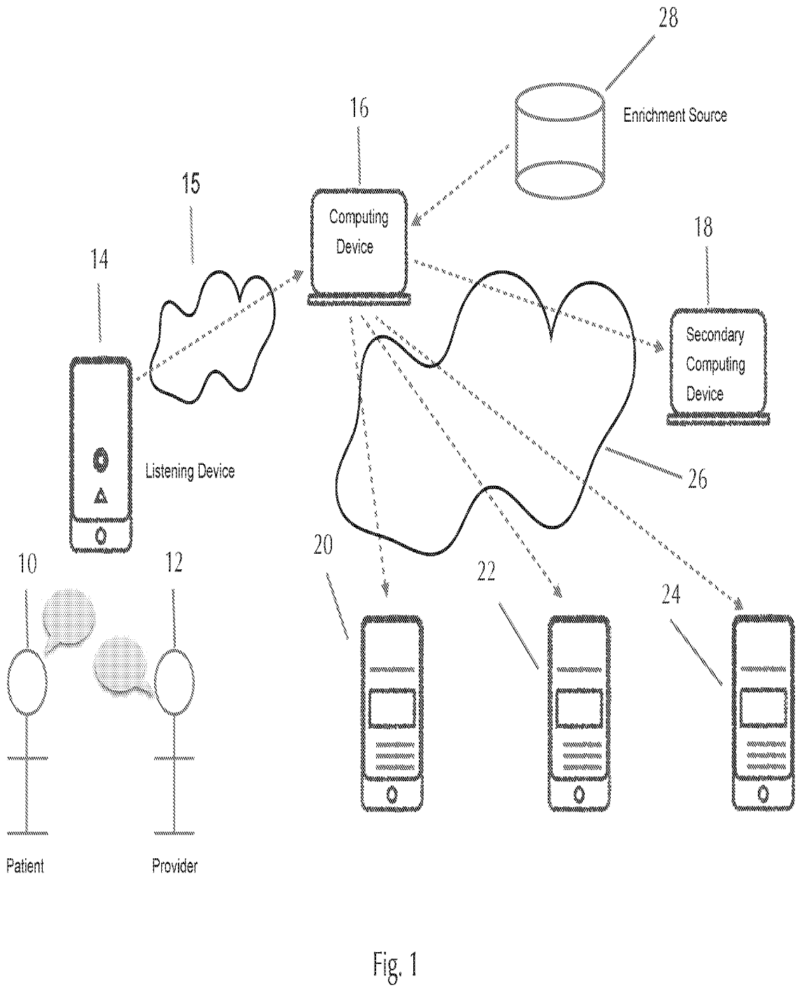

FIG. 1 illustrates the full system and major parts/components according to one embodiment. Typically a patient 10, or a provider 12, has a mobile device 14, configured to listen to an interaction between the patient and the provider, and record the interaction thereby generating raw information, and transmits the raw information to a primary computing device 16. In some embodiments, the raw information is automatically and immediately transmitted to the computing device 16. In other embodiments, the raw information is manually transmitted by either the provider or the patient to the primary computing device 16. In some embodiments, the raw information is automatically extracted by the primary computing device 16. In some embodiments the mobile device 14 and primary computing device 16 are configured to be on the same physical device, instead of separate devices. The embodiments of the system may include, or be capable, of accessing a data source 28, which can have stored thereon information useful to the primary computing device's 16 function of interpreting the received raw information from the mobile device 14, and/or adding data and/or editing the raw information based on the interpretation of the raw information thereby generating an output information. The system may also interface with secondary computing and mobile devices 18, 20, 22 and 24, which can be configured to receive and/or transmit information from the primary computing device 16. In some embodiments, the mobile device 14, the primary computing device 16, and the data source 28 are configured to be on the same physical device, instead of separate devices.

The computing devices, e.g. a primary computing device, are likely to change quickly over time. A task done on computer server hardware today will be done on a mobile device or something much smaller in the future. Likewise, smart mobile devices that are commonly in use at the time of this writing are likely going to be replaced soon by wearable devices, devices embedded in the body, nanotechnology and other computing methods. Different user interfaces can be used in place of a touch screen. Embodiments using other user interfaces are known or contemplated such as voice, brain-computer interfaces (BCI), tracking eye movements, tracking hand or body movements, and others. This will provide additional ways to access the output information generated by the embodiments disclosed herein. The primary computing device 16 is described herein as a single location where the main computing functions occur. However, computing steps such as analysis, extraction, enrichment, interpretation and others can also happen across a variety of architectural patterns. These may be virtual computing instances in a “cloud” system, they can all occur on the same computing device, they can all occur on a mobile device or any other computing device or devices capable of implementing the embodiments disclosed herein.

Embodiments of the system are capable of capturing an extended interaction between a patient and a provider using the mobile device 14. The interaction can be captured depending upon the type of interaction such as a recording, an audio, a video, and/or textual conversation such as online chat. The captured interaction is input data stream. In various embodiments of the disclosed system, the mobile device 14 is typically configured to transmit the input data stream to the primary computing device 16 as raw information for interpretation by the primary computing device 16 using HIPAA-compliant encryption. In some embodiments of the disclosed system, the raw information is typically transmitted across the Internet or other network 15 as shown in FIG. 1, but it may also be stored in the memory of the mobile device 14 and transferred to the primary computing device 16 by other means, such as by way of a portable computer-readable media. Transmission of raw information can be accomplished by means other than over the Internet or other network. This can happen in the memory of a computing device if the steps occur on the same device. It can also occur using other media such as a removable memory card. Other future means of data transmission can likewise be used without changing the nature of the embodiments disclosed herein.

Security measures are used to authenticate and authorize all users' (such as patient, provider, and/or users) access to the system. Authentication (determining the identity of patient, provider, and/or users) can be done using standard methods like a user name/password combination or using other methods. For example, voice analysis can be used to uniquely identify a person to remove the need for “logging in” and handle authentication in the course of normal speech. Other biometric authentication or other methods of user authentication can be used.

In some embodiments, the system detects the start of the interaction by way of the patient controlled mobile device 14, and the location services are subject to privacy controls determined by the patient. But the detection of the interaction can be done in a variety of ways. One example is by using location detection, for example, with location services in a mobile device such as GPS or beacons. Another example is by scanning the patient or provider's calendar for likely patient/provider appointments.

After receiving the raw information, the primary computing device 16 interprets the raw information and identifies and extracts relevant content therefrom. The primary computing device 16 can comprise any suitable device having sufficient processing power to execute the necessary steps and operations. The primary computing device can include, but is not limited to, desktop computers, laptop computers, tablet computers, smart phones and wearable computing devices, for instance. The primary computing devices are likely to change quickly over time. A task done on computer server hardware today will be done on a mobile device or something much smaller in the future. Likewise, smart mobile devices that are commonly in use at the time of this writing are likely going to be replaced soon by wearable devices, devices embedded in the body, nanotechnology and other computing methods. In various embodiments, the primary computing device is connected to a network 26 or 15, such as the Internet, for communicating with other devices, for example, device 14, 18, 20, 22, and 24 and/or database 28. The primary computing device in some embodiments can include wireless transceivers for directly or indirectly communicating with relevant other associated mobile and computing devices.

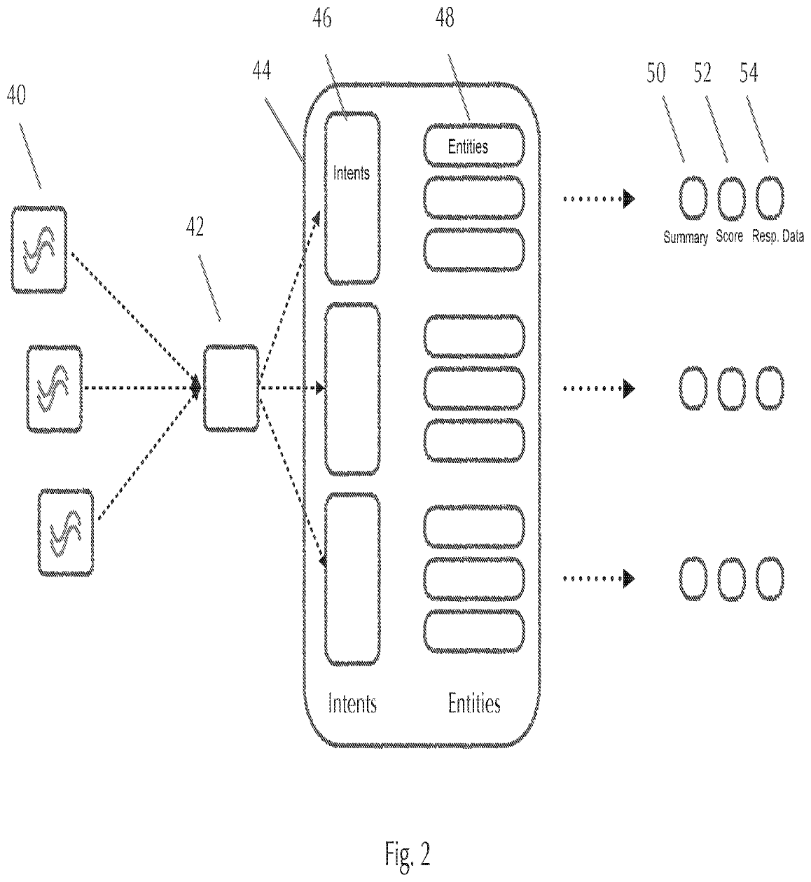

After receiving and storing the raw information on the primary computing device's 16 memory, the primary computing device 16 interprets the raw information and obtains relevant information therefrom adding additional content as warranted. The process is described with reference to FIG. 2. The use of a conversion module 42, and artificial intelligence module 44, as base technologies in the primary computing device 16, are well known to those with skill in the art of artificial intelligence software techniques.

In some embodiments of the disclosed system, the raw information is generated by the device 14 from the input data stream received by device 14. The input data stream can be a recording of the interactions between patient and provider. The raw information in the form of, e.g., audio files is transmitted to the primary computing device 16, in real time for interpretation.

The interpretation step is an implementation of artificial intelligence module designed to understand the context of these particular interactions between the patient and the provider, and/or the user. The artificial intelligence module 44 used in the primary computing device 16 is specially configured to be able to understand the particular types of interactions that occur between a provider and a patient as well as the context of their interaction. The interaction that happens between a patient and a provider is different from other types of typical interactions and tends to follow certain patterns and contain certain information. Further, these interactions are specific to different subsets of patient-provider interactions, such as within a medical specialty (e.g., cardiology) or related to a medical condition (e.g., diabetes), or patient demographic (e.g., seniors). Unlike other artificial intelligence systems, this artificial intelligence module 44 is configured to have a deep understanding of the patterns and content for the particular patient-provider subsets. In some subsets the engine can be configured to have multiple pattern understandings, for example, cardiology for seniors, and the like.

Intents 46 are generally understood in artificial intelligence module 44 to be recognition of what the interaction between the patient and provider means. The artificial intelligence module 44 uses Intents 46 in combination with a Confidence Score 52 to determine when a phrase in the raw information is relevant for inclusion in the output information such as in a summary or follow up action.

Entities 48 are the specific details in the interaction such as an address or the name of a medication.

The primary computing device 16 generates output information after extracting, and interpreting the raw information. The output information may include, but not limited to, the Intent 46, Entities 48 and other metadata required to be able to generate a summary, follow-up actions for the patient and or provider, and other meaningful information.

In one embodiment of the disclosed system, the primary computing device 16 operates as outlined in FIG. 2. In each case, the use of Expressions (not pictured) and Entities 48 train the system to be able to determine if a given audio file 40 of raw information matches an Intent 46 for a specific subset of a patient-provider interaction. The process of training the artificial intelligence module 44 depends on understanding the types of interactions that occur between a provider and a patient and match parts of that interaction to specific Intents 46. The types of interactions and information discussed varies greatly across medical specialties and a variety of other factors. The implementation of the training for the artificial intelligence module 44 can be done using techniques different than the one specified here. Intents, Entities and other specifics of the implementation can be replaced with similar terms and concepts to accomplish the understanding of the interaction. There are many algorithms and software systems used in the artificial intelligence field and the field constantly changes and improves. Other algorithms and software systems can be used to accomplish the interpretation and generation of output information comprising summaries & actions and other data from interactions between a patient, a provider and optionally a user.

Further, audio input 40 is fed to a conversion module 42 which translates the audio input 40 into a format that can be fed to the specially-trained artificial intelligence module 44 containing specially designed Intents 46 and Entities 48. The artificial intelligence module returns a response which comprises “Summary and Actions” 50 along with a Confidence score 52 to determine if a phrase heard as part of the interaction should be matched to a particular Intent 46 and other response data 54. The system creates unique output information comprising personalized “Summaries and Actions” 50 depending on the Intents 46 and Entities 48, along with other response data 54.

The extraction and interpretation of audio input by the primary computing device 16 is used to generate an output information that includes a summary of the interaction and generates follow up actions. This typically occurs in the same primary computing device 16, although these steps can also occur across a collection of computing devices, wherein the primary computing device 16 can also be replaced with a collection of interconnected computing devices. The audio input is a type of input data stream.

Many of the words said in the context of a patient-provider interaction include medical jargon or other complex terms. Enriching, as used herein, refers to adding additional information or context from a database 28 as shown in FIG. 1, so that the patient or user, can have a deeper understanding of medical jargon or complex terms. This enrichment occurs in the primary computing device 16. In this sense, the database is acting as an enrichment data source.

The database 28 can come from a variety of places, including (all must be done with a legal license to use the content): (1) API: information from application programming interfaces, from a source such as iTriage, can be used to annotate terms, including medications, procedures, symptoms and conditions, (2) Databases: a database of content is imported to provide annotation content, and/or (3) Internal: enrichment content may be created by users or providers of embodiments of the system, for example, the provider inputs data after researching the patient's specific issues.

Embodiments of the system may also provide methods for manually adding or editing output information. In some aspects, this modification is typically done by a patient advocate or a provider, or other person serving as a caregiver to the patient, or by the patient themselves. This often occurs in a secondary or remote computing device 18 as shown in FIG. 1. To accomplish this, the output information is transmitted from a primary computing device 16 to a secondary computing device 18 across the Internet or other network 26. The secondary computing device 18 can be any suitable computing device having processing capabilities. In some embodiments, the secondary computing device 18 may be the same device that serves as the mobile device 14. In other instances the secondary computing device 18 can be a remote computer, tablet, smart phone, mobile device or other computing device controlled by a caregiver or any other person who may directly or indirectly be involved in the care of the patient. Providers can manually enter notes, summaries and actions in addition to speaking to them. For example, discharge instructions may contain certain instructions that are the same for everyone, so those can be added to the summary and actions from the specific conversation.

All output information, including “Summaries and Actions” 50 and other response data 54, along with modifications made by a patient advocate or other persons using the secondary computing device 18, can be shared with others using a computing or a mobile device 24, subject to privacy controls. This can be accomplished by the patient using a computing or a mobile device 20, or by the provider using a computing or a mobile device 22. Data sharing may be facilitated by computing device 16, or in a peer-to-peer configuration directly between a computing or mobile device 20 or 22 to a computing or mobile device 24. Data is typically transmitted across the Internet or other network 26. In some instances, device 24 is present on the same physical device as device 14, instead of separate devices. Sharing can be done though a wide variety of means. Popular social networks such as Facebook and Twitter are one way. Other ways include group specific networks such as Dlife, group chat, text message, phone, and other means that have not yet been created. Other future sharing and social networking mechanisms can be used without changing the nature of embodiments of the system.

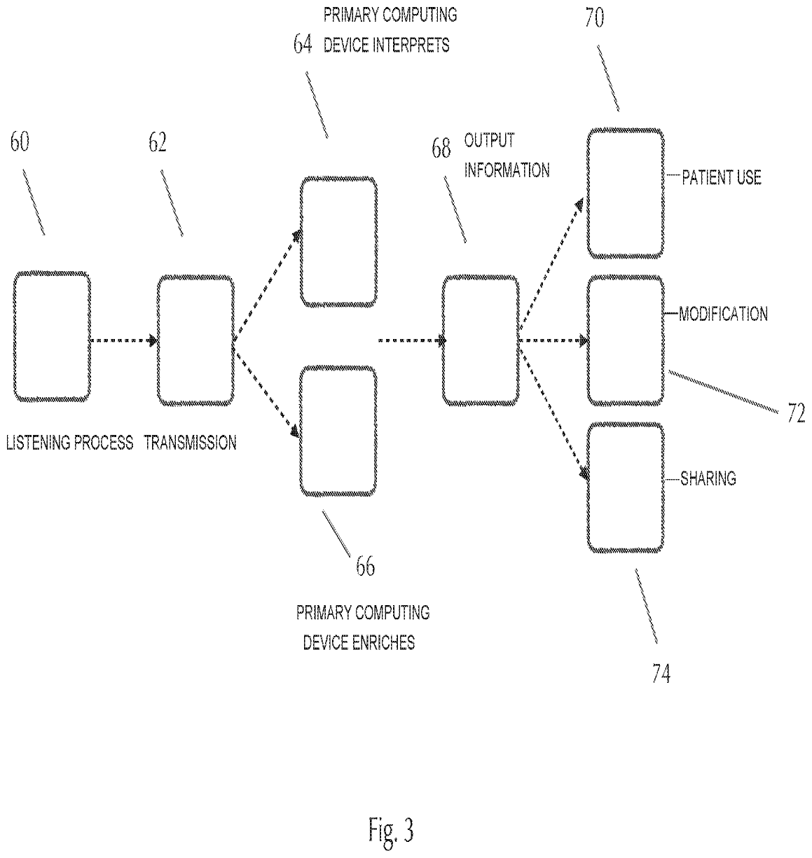

FIG. 3 shows a flow chart of one potential patient-provider interaction, using one embodiment of the disclosed system. This example illustrates one embodiment and does not represent all possible uses.

The listening process 60 may be initiated by the patient or by the provider typically by touching the screen of the mobile device 14 and, speaking to the mobile device 14. Alternatively, the listening process 60 is automatically started based on sensing or a timer. As described in the Sensing step above, the embodiments of the system may automatically detect that the patient appears to be in a situation when a clinical conversation may occur and prompt the patient or the provider to start the listening process, or it may start the listening process itself. This is particularly useful if the mobile device 14 is a wearable device or other embedded device without a user interface. This sensing reduces the need for the patient to remember to engage the system to start the listening process. In one example, the sensing is triggered by a term or phrase unique to the patient-provider interaction.

The embodiments of the system may give feedback about the quality of the recording via an alert to the mobile device 14, to give the participants the opportunity to speak louder or stand closer to the listening device.

The interaction between the patient and the provider is transmitted 62 to the primary computing device 16. The primary computing device 16 interprets the interaction and obtains meaningful information 64 and enriches with additional information 66 from the database 28 and generates the output information 68. The output information 68 includes a summary that contains the most important aspects of the interaction so that this information is easily available for later reference. This summary can be delivered to the provider, the patient, other caregivers or other people as selected according to the privacy requirements of the patient. This saves the provider from having to manually write the patient-provider visit summary, and ensures that the patient and provider have the same understanding of their interaction as well as provides expected follow up actions.

The output information 68 that includes summary and actions are transmitted to secondary computing devices used by patients, providers and other users. Output information includes a summary, follow-up actions for the patient and/or provider, and other meaningful information that can be obtained from the raw information. The system alerts the patient, and other users of the system, about information or actions that need attention, using a variety of methods, including push notifications to a mobile device. For example, based on the provider asking the patient to make an appointment during their interaction, the system may generate a calendar reminder entry to be transmitted to the calendar input of the patient's computing or mobile device 20. Or the system may generate a reminder to be transmitted to the patient on their mobile device. In some instances, device 20 is present on the same physical device as device 14, instead of separate devices.

While using and managing the output information 68 which includes summary, actions and other information, the patient can select (e.g. tap or click) to get background information and other research provided by the system to give them a deeper understanding of the results of the conversation analysis. For example, if the provider recommends that the patient undergo a medical procedure the system automatically gathers information about that procedure to present to the patient. This information could include descriptions, risks, videos, cost information and more. This additional information is generated in the primary computing device 16 and transmitted to secondary computing devices 20, 22, and/or 24.

Patients can use 70 the output information 68 for a variety of things including reminders, reviewing summary notes from the office visit, viewing additional information, sharing with family, and many other like uses.

Providers can make additional edits and modifications 72 to the output information 68. To augment the output information 68 that is generated automatically, the system provides a method for manually adding or editing information in the interpretation results. This modification 72 may be done by, for example, a patient advocate or other party acting on behalf of the patient or by the patient themselves.

Patients and other users with the appropriate security access can share 74 the output information 68 with family and other care givers or other people with the appropriate security access. The patient may choose to securely share parts of the output information 68 such as the summary, actions, and other information with people that the patient selects including family, friends and/or caregivers. To do this securely, data is encrypted in the primary computing device 16 and any secondary computing devices and transmitted over the Internet or other network 26 to a secondary computing device 24 possessed by the family, friends or caregivers. Sharing through popular social networking services is enabled by sharing a de-identified summary with a link to access the rest of the information within the secure system.

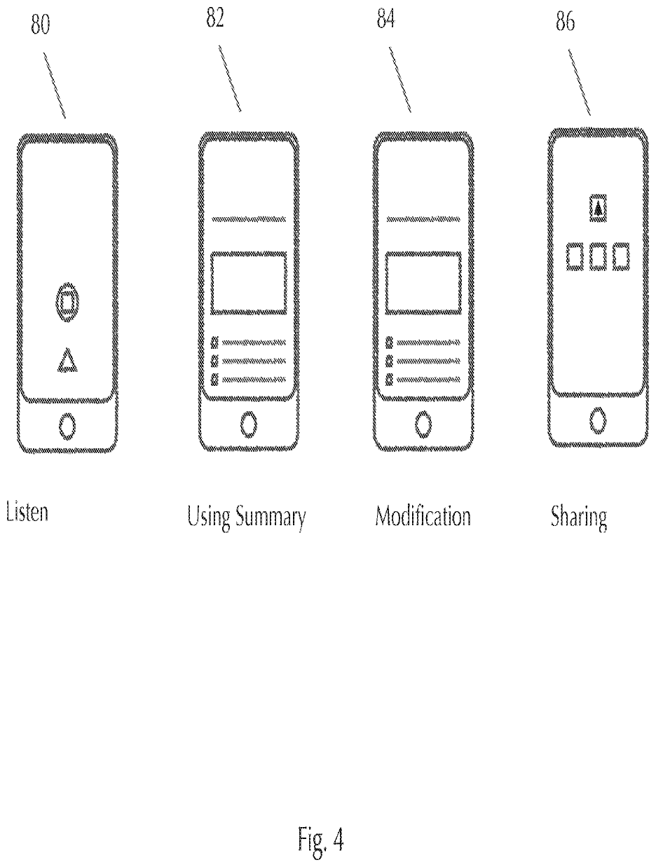

FIG. 4 illustrates a series of possible screen mockups for listening (including sensing) 80, using Summary and Actions 82, modification (by provider) 84, and sharing (with family, caregivers) 86 according to an embodiment of the disclosed system.

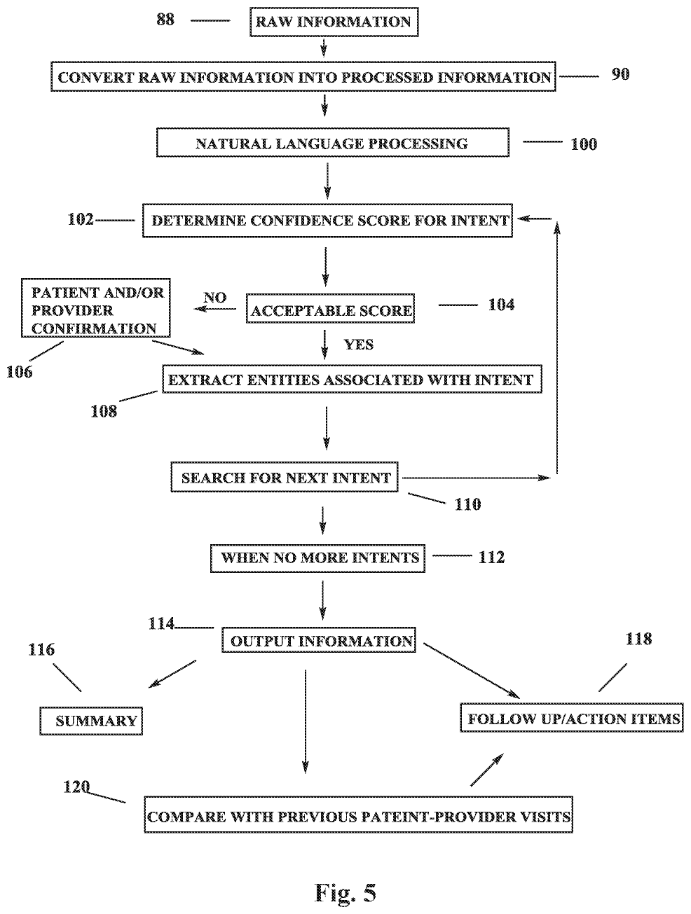

FIG. 5 illustrates a flow diagram for intents and entities according to one aspect of the disclosure.

In FIG. 5, after a patient-provider interaction, raw information 88 is generated and interpreted. The raw information is converted by a conversion module into a processed information 90. During the interpretation of the processed information the natural language processing techniques 100 are applied against the processed information to structure the processed information, look for intents relevant to the patient and extract other meaning from the information. The natural language processing techniques are part of the artificial intelligence module that also comprise other artificial intelligence techniques. As noted above, intents are meaning in language identified by the artificial intelligence module based on the context of the interaction between a patient, a provider and/or a user. The artificial intelligence module may be trained with intents and it may also determine intents to look for as it learns. For example, a generalized intent can include words and phrases like: physical therapy, workout, dosage, ibuprofen, and the like, as well as intents specific to the patient's needs, for example, the patient's daughter's name, patient's caregiver availability, known patient drug allergies, and the like. A confidence score 102 is applied against the intent to identify whether the intent has been applied within the processed information and other decisions made by the artificial intelligence are scored and highlighted to facilitate faster human review and confirmation by patient, provider or other reviewers when necessary. A sliding scale can be attached to each intent, for example, intents with lower safety concerns may have a lower confidence score requirement as compared to a drug dosage, where the confidence score would be higher. Where an intent fails it's confidence score, a question may be submitted to both patient and provider to confirm intent 106. Review and confirmation by patients, providers and/or reviewers also serve to train the artificial intelligence module to be more accurate in the future and build new skills. Such confirmatory queries may be submitted to the user's computing device, or may be queried from the listening device during the interaction. Where an intent is deemed acceptable 104, one or more entities 108 is applied to the intent. Entities are extracted from the content of the integration information related to the intent. For example, in the case of a ‘instruct to take meds’ intent, entities may include dosage, frequency and medication name. Then the processed information is searched again for the next intent 110 and the analyses starts again to apply entities. Once the entirety of the processed information is analyzed, i.e. all intents in the processed information have been analyzed 112, an output information 114 is generated comprising the summary 116 and follow up/action items 118. The output information 112 can be compared with earlier output information for the particular patient such as previous patient provider visit 120 to populate follow up/action items 118. For example, visits may be compiled to compare intents and entities over the course of two or more interactions to identify trends, inconsistencies, consistencies, and the like. In addition, comparisons can provide the patient and provider trends in the data, for example, the patient's blood pressure over the previous year, weight over the previous year, changes in medication, over the previous year. As above, follow-up actions can be built into the flow diagram.

In still other embodiments, output information is saved for each patient-provider visit. As additional visits occur, the output information may be compared to previous visit output information to identify useful rends, risk factors, consistencies, inconsistencies, and other useful information. In some embodiments, the patient and provider review the previous one or more output information at the new patient-provider interaction. Further, the output information from a series of patient-provider interactions can be tied together, for example, to provide the patient with his or her blood pressure chart and/or trends over the course of a year.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not intended to limit the scope of the invention. These examples are specific instances of the primary computing device's analysis operations. The implementation of this invention can contain an arbitrary number of such scenarios. The Expressions in each example illustrate phrases that would match to the Intent in that example.

### Example 1

The “pharmacy” Intent listens for provider/patient conversation about the patient's pharmacy according to one embodiment of the disclosed system.

| Intent | Pharmacy |
|---|---|
| Expressions | Question from the provider: “Which pharmacy do you |
| | use?” |
| | Answer from the patent: “We use the Walgreens at 123 |
| | Main Street.” |
| Entities | /pharmacy_name |
| | /address |
| Confidence | 0.725 |

(Expression) Doctor asks “Which pharmacy do you use?” and the patient replies “We use the Walgreens at 123 Main Street.”

(Intent) The primary computing device 16 extracts audio input and processes this conversation and analyzes it, recognizing that it matches a particular Intent, such as “pharmacy”.

(Entity) It identifies “Walgreens” as a place and “we” as a group of people, in this case the patient's family.

(Confidence) The primary computing device 16 analyzes the conversation and matches this particular sentence to the intent and returns a confidence score 52 along with the other information. If the confidence is high enough, it identifies the sentence or phrase as being related to this

Based on the analysis in this example, the primary computing device will generate an output information that will have at least the following attributes: record for the patient that the prescription was sent to the Walgreens at 123 Main Street; create a reminder to pick up the prescription; include a map showing the location and driving direction; enrich the results with additional information, for example details about the medication.

### Example 2

The “instruct exercise” Intent listens for provider instructions related to the exercise or physical therapy regimen of the patient according to one embodiment of the disclosed system.

| Intent | instruct_exercise |
|---|---|
| Expressions | “Please get to the gym at least 3 times per week” |
| | “Please workout 3 times per week” |
| | “Overall things are going pretty well but I'd like you to |
| | start working out 3 times a week and then come back to see |
| | me in a month.” |
| | “Things are good but I would like you to start working |
| | out 3 times per week” |
| | “workout twice a week” |
| | “I'd like you to exercise 4 times per week” |
| Entities | /instruction |
| | /frequency |
| Confidence | 0.892 |

Based on the analysis in this example, the primary computing device 16 will generate an output information that will have at least the following attributes: enter the instruction to exercise into the visit summary; create a reminder to exercise and send the reminder to the patient's mobile device recurring on the frequency indicated in the Entity (e.g. 3 times per week).

### Example 3

The “instruct to take meds” Intent listens for provider instructions related to proper medication adherence for the patient according to one embodiment of the disclosed system.

| Intent | Instruct_to_take_meds |
|---|---|
| Expressions | “Since your daughter is under 35 pounds you can give her 5 |
| | milliliters of Ibuprofen every 6 hours” |
| | “Since your daughter is under 35 pounds you can give her 5 |
| | millileters of Advil every 6 hours” |
| | “I'd like you to increase your Lexapro from 10 to 20 mg |
| | per day for another two weeks” |
| | “I'd like you to increase your Lexapro from 10 to 20 |
| | milligrams per day for another two weeks” |
| Entities | /dosage |
| | /frequency |
| Confidence | 0.842 |

Based on the analysis in this example, the primary computing device will generate an output information that will have at least the following attributes: enter the instruction to exercise into the visit summary; create a reminder and send the reminder to the mobile device of the patient to take the medication indicated on the frequency indicated in the Entity.

### Example 4

Description of an artificial intelligence module usage scenario according to one embodiment of the disclosed system.

A provider (doctor), patient, user (e.g., family member of the patient) discuss patient's injured wrist. The patient describes to the provider that she injured her wrist about three weeks ago ad it's been hurting with a low-grade pain since then. The doctor inquires the patient with some general health questions, including but not limited to, her mental and emotional state. The provider order preliminary diagnostic tests, including but not limited to, x-ray.

The provider informs the patient that the x-ray was negative and that she has a bad sprain. The provider prescribes her 800 mg of ibuprofen b.i.d. (twice daily) for one week and advise her to make a follow-up appointment after three weeks.

In embodiment of the system, the system listens to the provider-patient conversation and captures provider's visit notes. The system put parts of the conversation into different sections as appropriate. For example in the chart notes there is a history section, an exam section and an assessment section. The system automatically puts the discussion of the patient's general state of health and mental emotional state into the history section. The system automatically puts the doctor's comments about the x-ray into the exam section and comments about the treatment plan into the assessment section. The system also generates a summary of the patient-provider conversation during the patient's visit.

The system automatically creates two patient instructions—one for the patient to take 800 milligrams of ibuprofen two times daily for one week and the other for the patient to schedule a follow-up appointment after three weeks.

The summary, patient instructions and full conversation text are sent to the patient electronically. The patient now has this information for her own use and can share it with other people including family and caregivers. The system also enriches the information by adding further details that may be useful to the patient. For example, the patient can tap on the word ibuprofen and get full medication information including side effects.

The summary, patient instructions and full conversation text is also sent to the provider and the visit chart notes are inserted into the electronic health record system.

### Example 5

Output information according to one embodiment of the disclosed system.

Current Visits Record A Visit

Feb. 14, 2016
Patent A
Visit conversation transcript appears here

### Example 6

Output information according to one embodiment of the disclosed system.

Review

Visit Detail
Edit Save to Electronic Health Record
Back
New Visit
Visit Date/Time: 04/25/2016, 10:57 PM
UTC
Visit Name: Friday afternoon visit
Patient Name: Patient A
History:
Patient A has been having problems with his right wrist for the last 3
weeks resulting from pickup football game
Exam:
Did physical exam and x-rays
Assessment:
He has a sprained wrist and I prescribed 40 mg of Advil to take 2 times
per day for pain

### Example 7

Output information according to one embodiment of the disclosed system.

Review

Patent Name: Patient A
History:
Patient A has been having problems with his right wrist for the last 3
weeks resulting from pickup football game
Exam:
Did physical exam and x-rays
Assessment:
He has a sprained wrist and 1 prescribed 40 mg of Advil to take 2 times
per day for pain
General Comments:
Patent A seems to be in good spirits overall
Patent Instructions:
Take 40 mg of Ibuprofen 2 times daily
Full Conversation:
Patent A seems to be in good spirit overall #history Patient A has been
having problems with his right wrist for the last 3 weeks resulting
from pickup football game #exam did

What is claimed is:

1. A computer-implemented system, comprising:
a primary computing device having a computer memory storage module configured to store executable computer programming code, and a computer processor module operatively coupled to the computer memory storage module of the primary computing device, wherein the computer processor module is configured to execute the computer programming code to perform the following functions:
receiving an input data stream from an interaction between at least one patient and at least one provider;
extracting the received input data stream to generate a raw information;
interpreting the raw information by converting the raw information using a conversion module to produce a processed information;
providing a database having medical information for enriching the processed information;
analyzing the processed information using an artificial intelligence module wherein the artificial intelligence module uses an intent in combination with a confidence score to determine one or more entities to include an output information;
generating the output information based upon the analysis of the processed information; and
providing instructions to the at least one patient based on the output information.

2. The system of claim 1, wherein analyzing the processed information further comprises: understanding the content of the processed information.

3. The system of claim 1, further comprising sharing the output information with at least the provider, and/or the user.

4. The system of claim 1, further comprising updating a patient record in an electronic health records system based upon the processed information or the output information.

5. The system of claim 1, wherein the output information is further modified by the provider.

6. The system of claim 1, wherein the detection of the interaction is automatic or manually initiated by one of the provider or patient.

7. A non-transitory, tangible computer readable medium storing instructions that when executed electronically by one or more computer processors generate personalized instructions by performing the following steps:
receiving an input data stream from an interaction between at least one patient and at least one provider;
extracting the received input data stream to generate a raw information;
interpreting the raw information by converting the raw information using a conversion module to produce a processed information;
providing a database having medical information for enriching the processed information;
analyzing the processed information using an artificial intelligence module wherein the artificial intelligence module uses an intent in combination with a confidence score to determine one or more entities to include an output information;
generating the output information based upon the analysis of the processed information; and
providing instructions to the at least one patient based on the output information.

8. The medium of claim 7 wherein analyzing the processed information further comprises: understanding the content of the processed information.

9. The medium of claim 7, further comprising sharing the output information with at least the provider, and/or the user.

10. The medium of claim 7, further comprising updating a patient record in an electronic health records system based upon the processed information or the output information.

11. The medium of claim 7 wherein the output information is further modified by the provider.

12. The medium of claim 7 wherein the detection of the interaction is automatic or manually initiated by one of the provider or patient.

13. A computer-implemented method comprising:
receiving an input data stream from an interaction between at least one patient and at least one provider;
extracting the received input data stream to generate a raw information;
interpreting the raw information by converting the raw information using a conversion module to produce a processed information;
providing a database having medical information for enriching the processed information;
analyzing the processed information using an artificial intelligence module wherein the artificial intelligence module uses an intent in combination with a confidence score to determine one or more entities to include an output information;
generating the output information based upon the analysis of the processed information; and
providing instructions to the at least one patient based on the output information.

14. The method of claim 13 wherein analyzing the processed information further comprises: understanding the content of the processed information.

15. The method of claim 13, further comprising sharing the output information with at least the provider, and/or the user.

16. The method of claim 13, further comprising updating a patient record in an electronic health records system based upon the processed information or the output information.

17. The method of claim 13 wherein the output information is further modified by the provider.

18. The method of claim 13 wherein the detection of the interaction is automatic or manually initiated by one of the provider or patient.
